(19) United States
(12) Patent Application Publication
(10) Pub. No.: US 2026/0090822 A1
(43) Pub. Date: Apr. 2, 2026

(54) Selectable Angle Needle Guide for use with Imaging Probes

(71) Applicant: David Kinas, Miami, FL (US)

(72) Inventor: David Kinas, Miami, FL (US)

(21) Appl. No.: 18/904,220

(22) Filed: Oct. 2, 2024

(51) Int. Cl. A61B 17/34

(52) U.S. Cl. CPC A61B 17/3403

(57) ABSTRACT

A needle guide assembly is provided having a body section and a needle guide body. The body section is configured to engage the head of an ultrasound or similar imaging probe. The needle guide body includes a pair of parallel leg members that extend from the body section. The leg members form a channel therebetween that is sized and configured to allow a needle to be inserted therethrough and into the tissue of a patient under visual observation by the imaging probe.

### PRIOR ART

A variety of approaches have been developed to assist in the insertion of a needle into the body of a patient in order to access a subcutaneous target.

In U.S. Pat. No. 4,838,506 granted to Cooper, a holder for a needle that is used in combination with an ultrasound probe is disclosed. In this approach, a separate needle guide sleeve is provided to allow the user to insert a needle into the field of vision for the ultrasound probe. The orientation of the sleeve is movable in two dimensions relative to the ultrasound probe such that when the sleeve is oriented in the desired position relative to the ultrasound probe, the needle is inserted into the tissue of the patient.

In U.S. Pat. No. 9,974,516 granted to Orome et. al., a selectable needle guide assembly is disclosed wherein the guide assembly includes multiple guide channels for inserting a needle at a desired angle. Additional guide assemblies are disclosed for differing needle sizes. As with the Cooper patent, the needle assembly of this patent requires the placement of the needle through a sleeve or similarly restrictive element prior to insertion into the skin of the patient.

In U.S. Pat. No. 11,998,386 granted to Lindekugel et. al., a support and structure cover for an ultrasound probe head is disclosed. In one of the embodiments disclosed in this patent, a notch shaped needle guide is provided. In this embodiment, the needle is positioned below the needle guide of the probe cap and then moved against the notch shaped needle guide during needle insertion. In another embodiment, the probe cap includes rails structures to allow the needle guide to be positioned at a desired position away from the probe cap and head of the ultrasound member.

Each of the forgoing references include restrictions on the movement of the needle during insertion into the tissue of the patient. With the sleeves or guide channels, the angle and path of the needle insertion must be selected and predetermined prior to the procedure. This can restrict the operator in making adjustments and redirections during the procedure. This approach also prohibits the user from removing the guide during the procedure while the needle is inserted through the skin and prevents the operator from the ability to slide over a catheter. With the notch configuration, the needle must be positioned beneath the end of the needle guide prior to insertion into the tissue of the patient limiting the operators ability to freely change the needle angle and redirect during the procedure after needle insertion. The distance in which the needle is inserted from the ultrasound probe much be set within the preset distance intervals prior to the procedure which can prohibit the ability to make easy and free adjustments to the distance in which the needle is inserted in relation to the ultrasound probe.

### SUMMARY OF THE INVENTION

Briefly summarized, embodiments of the present invention are directed to a needle guide assembly for inserting a needle into the body of a patient in order to access a an anatomic structure of interest such as a vessel, nerve, joint or fluid collection. In one embodiment, the needle guide assembly comprises a body section that is configured to at least indirectly and removably attach to an image producing device, such as an ultrasound probe. The needle guide body defines at least a first elongate guide channel that is open at one end and closed at the other end. In addition, other needle guide assemblies are disclosed that include a needle guide body having a desired width depending on the size of the needle intended to be used in the procedure. Furthermore, the needle guide assembly may be attachable to the transverse or longitudinal sides of the ultrasound probe, allowing both in plane and out of plane techniques to be utilized. Yet another approach allows for the use of interchangeable needle guide bodies depending on the desired size of the needle and another approach allows the user to insert the needle at an angle that is perpendicular to the body of the probe and generally parallel to the face of the probe. This versatility allows a variety of insertion angles into the patient's body and allows the user to insert the needle using the transverse/out of plane approach or the longitudinal/in plane approach in relation of the ultrasound probe.

The use of the guide channel allows the user the freedom to insert the needle at any desired angle while having the option to adjust angles and needle redirections during the procedure while ensuring that the needle is inserted on the desired plane relative to the ultrasound probe transducer beam allowing optimal needle visualization during the procedure. The guide channel allows the needle tip to be placed at multiple depths and target locations during the procedure which may be essential for a variety of procedures such as in regional anesthesia. Additionally, the use of the guide channel allows the user to lift the needle guide assembly and ultrasound probe from the skin surface of the patient and insert a catheter over the needle to the desired location that has been previously identified during placement of the needle. Also, the nature of the needle being aligned in the track of the needle guide, allows the user to pass guide wires without having to take the probe off the patient which requires two hands. One hand can hold the needle and the guide at the same time which is a fixed to the probe while the other hand passes the guidewire. Without this device you usually have to ditch the probe during guide wire insertion since it requires two hands and then place the probe back on after the procedure to visualize the guide wire. These types of procedures are not available in devices that use enclosed sleeves for the needle.

The present invention generally includes a body section that is configured to attach to head section of the ultrasound probe. The body section may be tailored for use on specific body configurations of an ultrasound probe such as flat transducer surface (linear array or phased array probe), curved transducer surface (curvilinear, or endocavitary probe) or other specialty ultrasound probes such as hockey stick style probes. The needle guide body may be integral with the body section or separately attachable to the body section. The guide channels are preferably sized for specific needle sizes and may include needle guide rulers to allow the user to quickly determine the distance from the midline of the ultrasound beam or labeled angle guides that provide references in aiding the operator with their needle insertion technique and approach. The labeled angle guide set at 45 degrees can assist the operator in reaching a desired depth target by utilizing pythagorean theorem and determining desired needle entry distance. Additionally, the nature of the long needle guide channel, specifically configurations which include the longitudinal and transverse guide channels in combination helps provide a wide footprint that stabilizes the probe in an optimal perpendicular orientation to the patient by limiting the rocking or fanning motion of the probe. This makes doing the procedure easier by making sure the probe is perpendicular during the procedure.

These and other features of embodiments of the present invention will become more fully apparent from the following description and appended claims or may be learned by the practice of embodiments of the invention as set forth hereinafter.

### BRIEF DESCRIPTION OF DRAWINGS

FIG. 1 is a side view showing the needle guide assembly of the present invention with a needle and a flat ultrasound probe;

FIGS. 2A and 2B are perspective views showing the needle guide assembly of the present invention showing multiple example needles, a linear array ultrasound probe, a needle guide ruler and a 45 degree needle guide;

FIG. 2C is a perspective view showing the needle guide assembly of the present invention separated from the needle;

FIG. 3 is a top view of an embodiment of the needle guide assembly of the present invention;

FIG. 4 is a side view of the embodiment of the needle guide assembly of the present invention shown in FIG. 3;

FIG. 5 is a bottom view of the embodiment of the needle guide assembly of the present invention shown in FIG. 3;

FIG. 6 is an end view of the embodiment of the needle guide assembly of the present invention shown in FIG. 3;

FIG. 7 is a cross-sectional view of the embodiment of the needle guide assembly of the present invention shown in FIG. 3;

FIG. 8 is a top view of an alternate embodiment of the needle guide assembly of the present invention;

FIG. 9 is an end view of the alternate embodiment of the needle guide assembly of the present invention shown in FIG. 8;

FIG. 10 is a cross sectional view of the alternate embodiment of the needle guide assembly of the present invention shown in FIG. 8;

FIG. 11 is a bottom view of the alternate embodiment of the needle guide assembly of the present invention shown in FIG. 8;

FIG. 12 is a perspective view of the alternate embodiment of the needle guide assembly of the present invention shown in FIG. 8;

FIG. 13 is a top view of an alternate embodiment of the needle guide assembly of the present invention;

FIG. 14 is a cross sectional view of the alternate embodiment of the needle guide assembly of the present invention shown in FIG. 13;

FIG. 15 is a perspective view of an alternate embodiment of the needle guide assembly of the present invention;

FIG. 16 is a top view of the alternate embodiment of the needle guide assembly of the present invention shown in FIG. 15;

FIG. 17 is a bottom view of the alternate embodiment of the needle guide assembly of the present invention shown in FIG. 15;

FIG. 18 is an end view of the alternate embodiment of the needle guide assembly of the present invention shown in FIG. 15;

FIG. 19 is a top view of an alternate embodiment of the needle guide assembly of the present invention;

FIG. 20 is a bottom view of the alternate embodiment of the needle guide assembly of the present invention shown in FIG. 19;

FIG. 21 is a side view of the alternate embodiment of the needle guide assembly of the present invention shown in FIG. 19;

FIG. 22 is a top view of an alternate embodiment of the needle guide assembly of the present invention;

FIG. 23 is a bottom view of the alternate embodiment of the needle guide assembly of the present invention shown in FIG. 22;

FIG. 24 is an end view of the alternate embodiment of the needle guide assembly of the present invention shown in FIG. 22;

FIG. 25 is a perspective view of an alternate embodiment of the needle guide assembly of the present invention;

FIG. 26 is a side view of the alternate embodiment of the needle guide assembly of the present invention shown in FIG. 25;

FIG. 27 is a top view of the alternate embodiment of the needle guide assembly of the present invention shown in FIG. 25;

FIG. 28 is a bottom view of the alternate embodiment of the needle guide assembly of the present invention shown in FIG. 25;

FIG. 29 is an end view of the alternate embodiment of the needle guide assembly of the present invention shown in FIG. 25;

FIG. 30 is a perspective view of an alternate embodiment of the needle guide assembly of the present invention;

FIG. 31 is a bottom view of the alternate embodiment of the needle guide assembly of the present invention shown in FIG. 30;

FIG. 32 is a perspective view of an alternate embodiment of the needle guide assembly of the present invention;

FIG. 33 is a bottom view of the alternate embodiment of the needle guide assembly of the present invention shown in FIG. 33;

FIG. 34 is a perspective view of an alternate embodiment of the needle guide assembly of the present invention;

FIG. 35 is a perspective view of the alternate embodiment of the needle guide assembly of the present invention shown in FIG. 34;

FIG. 36 is a perspective view of the alternate embodiment of the needle guide assembly of the present invention shown in FIG. 34;

FIG. 37 is a perspective view of an alternate embodiment of the needle guide assembly of the present invention;

FIG. 38 is a side view of the alternate embodiment of the needle guide assembly of the present invention shown in FIG. 37;

FIG. 39 is an exploded perspective view of the alternate embodiment of the needle guide assembly of the present invention shown in FIG. 37;

FIG. 40 is a diagrammatic side view of the alternate embodiment of the needle guide assembly of the present invention shown in FIG. 37; and

FIG. 41 is a diagrammatic perspective view of an alternate embodiment of the needle guide assembly of the present invention shown in FIG. 37.

### DETAILED DESCRIPTION OF THE INVENTION

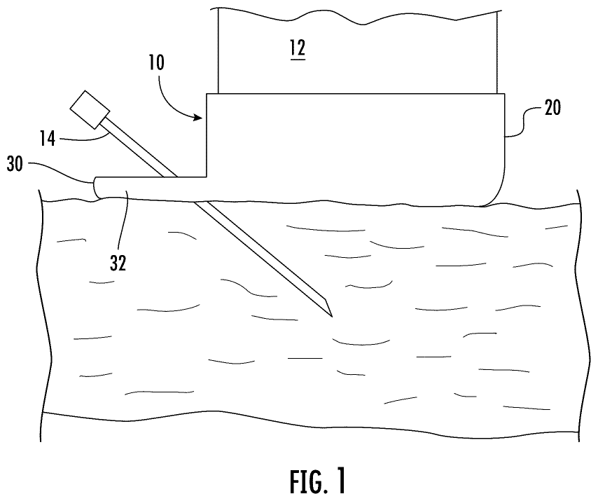

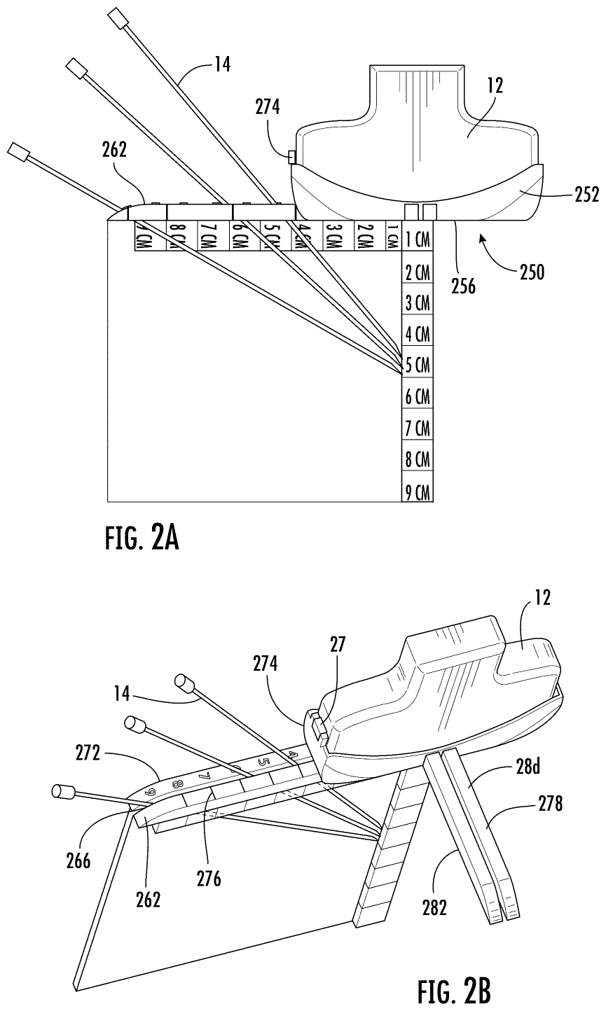

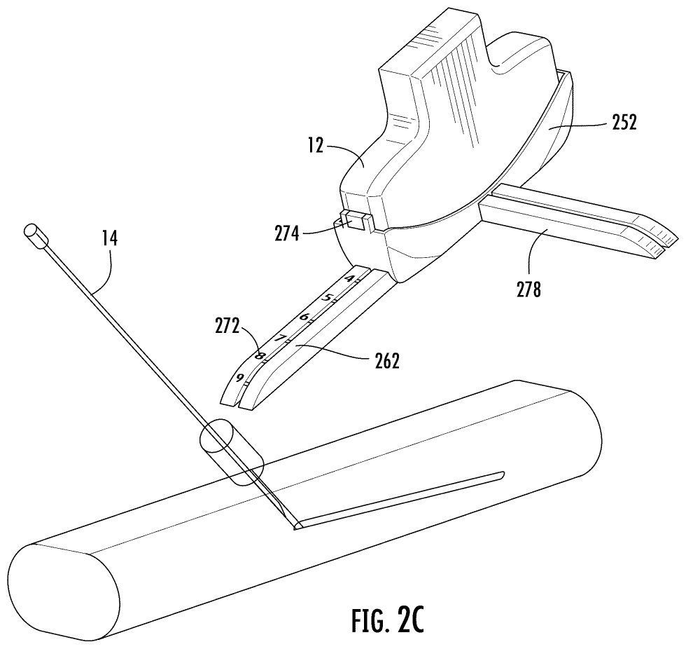

As illustrated generally in FIGS. 1, 2A and 2B, the embodiments of the present invention are directed to a needle guide assembly for inserting a needle 14 into the body of a patient to access a subcutaneous target, such as a vessel, nerve, joint or fluid collection. As shown in FIGS. 1, 2A and 2B, the ultrasound probe 12 may have a flat imaging surface or a curvilinear surface. Additionally, the needle guide assembly may be releasably attached to the ultrasound probe 12 by engaging the indicator notch of the ultrasound probe 12. Ultrasound probes 12 typically include the probe indicators to allow the user to determine what is the left and right side on the ultrasound machine screen (not shown).

As shown in FIGS. 2A and 2B, a needle guide ruler 272 is located along the path of the needle guide will allow the operator to know how far away the needle entry will be in relation to the center of the ultrasound probe. By using Pythagorean theorem the operator can best estimate the trajectory of the needle path at 45 degrees (ie: If the target is 4 cm deep, by entering the skin at 4 cm on the needle guide at 45 degrees the needle will be at 4 cm depth at the center of the ultrasound probe. Furthermore, an angle guide 276 may be provided along the needle guide to provide guidance for the angle of entry for the needle. Additionally, FIGS. 2A and 2B illustrates the ability of the user to control the needle position along the y and z axis of the needle guide assembly. FIGS. 2A and 2B provide multiple illustrative needle positions to show relative needle positioning for shallow, medium and steep needle entry. Each of these features are described in more detail below as these features are described with respect to the embodiments described herein.

The preferred use of the present invention involves the use of an ultrasound probe 12 or similar imaging device to visualize a target or organ of interest. Initially, the medical personnel use the ultrasound probe to identify the structure of interest. Next, the user selects a needle having the desired length and size depending on the type of procedure and area of interest. The user then attaches the needle guide assembly to the ultrasound probe. When the user locates the area of interest again, the needle is inserted into the guide channel of the guide assembly and the desired angle of approach is selected. As the needle is inserted into the tissue, the angle of attack can be adjusted based on the visualization provided by the ultrasound probe. The guide channel allows the user to insert the needle at multiple desired angles such that the needle penetrates the tissue at multiple desired depths while ensuring that the needle is inserted on the desired plane relative to the ultrasound probe and above or below various other structures in the tissue of the patient. The present invention is also longer than many available devices allowing needle entry to be as close or as far away as the guide allows. In practice, especially with deep cannulation and nerve blocks, needle entry multiple centimeters away from the probe is important to allow the needle to get to its targeted depth while maintaining a shallower angle for better needle visualization. The entry angle is also important if you are trying to place the needle underneath neurovascular structures such as when the needle is too close to the probe. If the needle angle is too steep the user may not be able to effectively place the needle under the target and needle visualization becomes difficult due to the limited ability to adapt the angle of insertion to the tissue and target. With the present design, an important feature of the present invention is that the user can enter the skin as far or as close as the user wants for more flexibility in the approach for the procedure while still staying in plane for visualization by the ultrasound probe. The ultrasound probe and needle guide system can also be moved dynamically closer or further from the needle with the needle is still located in the track allowing the operator to visualize either structures proximal or distal to the needle and furthermore adding the user more freedom for adjustability and approach during the procedure while keeping the needle in plane and in view.

FIG. 2C is illustrative of a further feature of the present invention wherein the needle guide assembly and ultrasound probe may be readily separated from a previously inserted ‘:?>the visualization provided by the ultrasound probe 12. The needle guide assembly and the ultrasound probe 12 may then be moved out of the way to allow a catheter to be inserted over the needle.

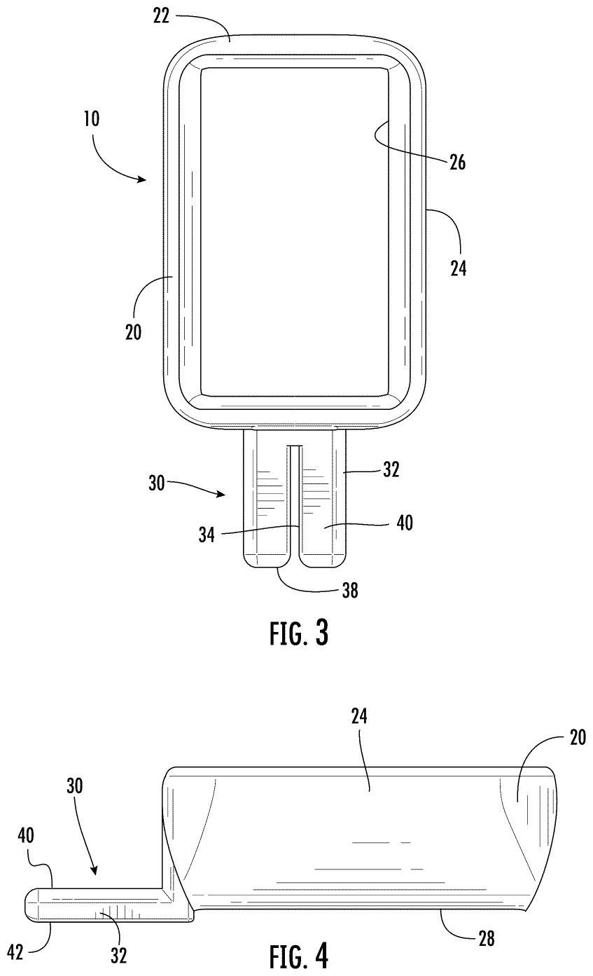

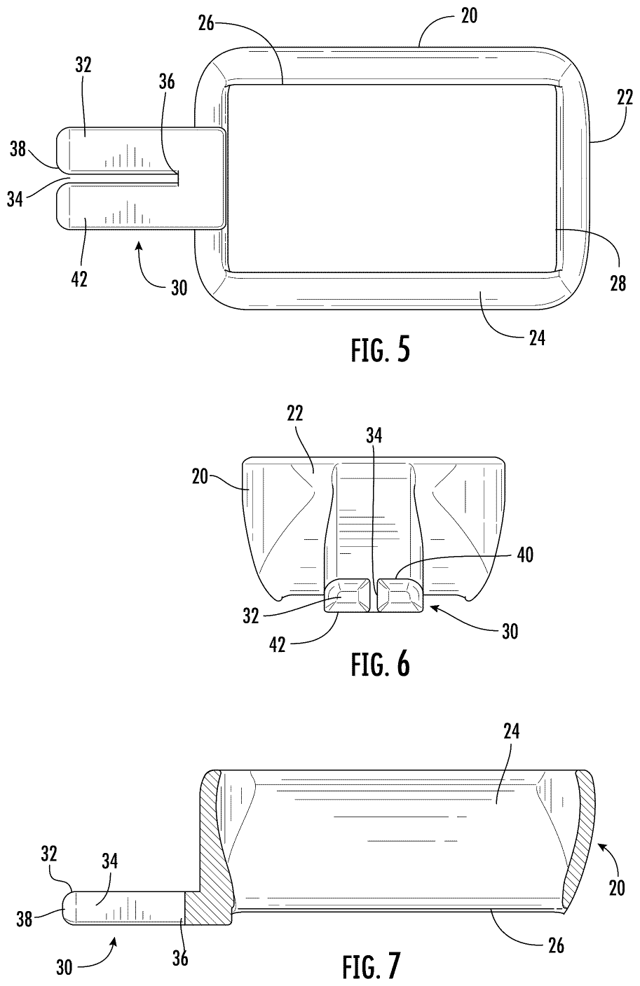

As shown in FIGS. 3-7, The needle guide assembly 10 of the present invention generally includes a body section 20 and a needle guide body 30. In a preferred form of the present invention, the body section 20 is generally rectangular in shape to conform to the shape of the ultrasound probe 12. The body section 20 includes a pair of shorter length sides 22 and a pair of longer length sides 24. The shorter sides 22 correspond to the lateral side of the ultrasound probe 12 and the longer sides 24 of the body section 20 correspond to the transverse side of the ultrasound probe 12. The body section 20 also includes a lip surface 26 that preferably extends along the entire lower surface of the ultrasound probe 12 while not covering or obscuring the ultrasound probe transducer face. The lip surface 26 engages the ultrasound probe 20 to allow the needle guide assembly 10 to be preferably aligned with the end of the ultrasound probe 12 for procedures where longitudinal access to the tissue of the patient is desired.

As shown, the needle guide body 30 is a generally U-shaped member that extends outwardly from the shorter side 22 of the body section 20. The needle guide body 30 includes a pair of elongate leg members 32 that extend generally from the midpoint of the shorter side 22 of the body section 20. The guide channel 34 extends between the leg members 32 and preferably has a width dimension that conforms to the desired needle diameter or size for use in the preferred procedure. The guide channel 34 starts at a first end 36 on the needle guide body 30 spaced a short distance from the shorter side 22 of the body section 20. The guide channel 34 extends from the first end 36 to an open second end 38 that is spaced apart from the body section 20. The height or depth of the guide channel 34 between the top surface 40 of the needle guide body 30 and the bottom surface 42 of the needle guide body 30 is selected to limit side to side or lateral movement of a needle 14 when a needle 14 is inserted into the guide channel 34. The lengthwise dimension of the guide channel 34 allows the needle 14 to be inserted at various angles along the y axis relative to the needle guide assembly 10 and tissue of the patient as discussed more fully below. In a preferred form of the present invention, the bottom surface 42 of the needle guide body 30 is generally aligned and positioned in line with the ultrasound probe transducer face which is generally inline with the bottom surface 28 of the body section 20.

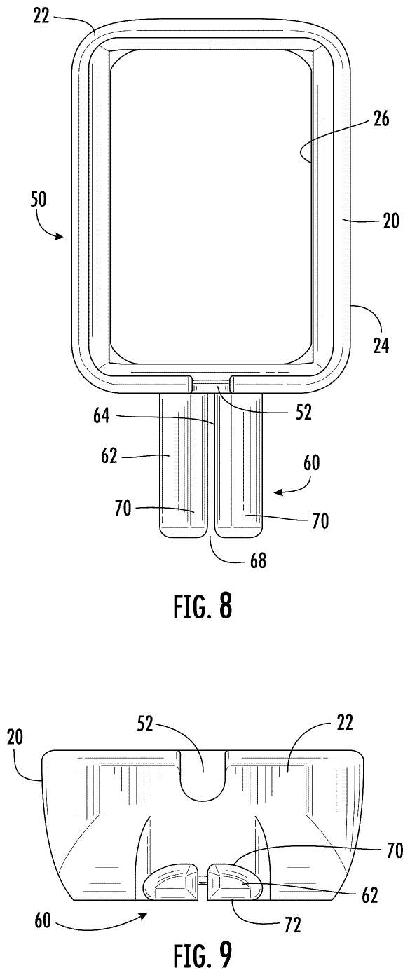

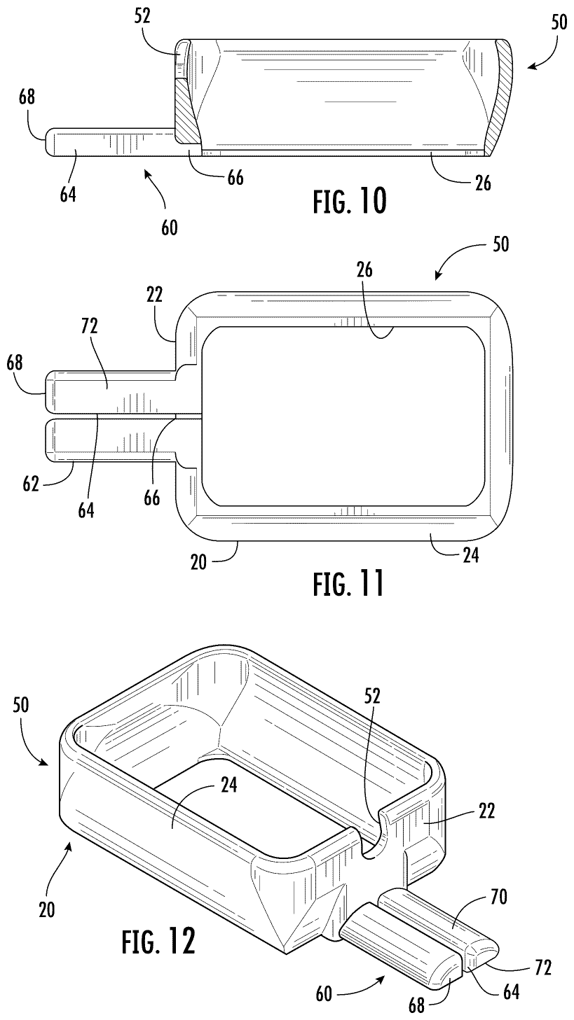

As shown in FIGS. 8-12, an alternate form of the needle guide assembly 50 of the present invention is disclosed. To assist in the understanding of the present invention, like numbers are used for like elements. The needle guide assembly 50 of this embodiment generally includes a body section 20 and a needle guide body 60. In a preferred form of the present embodiment, the body section 20 is generally rectangular in shape to conform to the shape of the ultrasound probe 12. The body section 20 includes a pair of shorter length sides 22 and a pair of longer length sides 24. The shorter sides 22 correspond to the lateral side of the ultrasound probe 12 and the longer sides 24 of the body section 20 correspond to the transverse side of the ultrasound probe 12. The shorter side 22 of this embodiment adjacent to the needle guide body 60 includes an upper semicircular slot 52 to allow for the passage of an ultrasound probe indicator 27 that is located on every ultrasound probe to basically let you know whats left and whats right on the ultrasound machine screen. Each manufacture and ultrasound probe may vary in the structural shape or design of the indicator 27 and therefore, the semicircular slot is designed to receive a variety of shapes of indicators 27 from the ultrasound probe 12 therethrough. The body section 20 also includes a lip surface 26 that preferably extends along the entire lower surface of the ultrasound probe 12. The lip surface 26 engages the ultrasound probe 12 to allow the needle guide assembly 50 to be preferably aligned with the end of the ultrasound probe 12 for procedures where longitudinal access to the tissue of the patient is desired.

As shown, the needle guide body 60 of this embodiment is a generally U-shaped member that extends outwardly from the shorter side 22 of the body section 20. The needle guide body 60 includes a pair of elongate leg members 62 that extend generally from the midpoint of the shorter side 22 of the body section 20. The guide channel 64 extends between the leg members 62 and preferably has a width dimension that conforms to the desired needle size for use in the preferred procedure. The guide channel 64 starts at a first end 66 on the needle guide body 30 below the shorter side 22 of the body section 20. This configuration of the first end 66 enables the user to use a shallower angle of entry for the needle 14 into the tissue of the patient. The guide channel 64 extends from the first end 66 to an open second end 68 that is spaced apart from the body section 20. The height or depth of the guide channel 64 between the top surface 70 of the needle guide body 60 and the bottom surface 72 of the needle guide body 60 is selected to limit side to side or lateral movement of a needle 14 when a needle 14 is inserted into the guide channel 64. In this embodiment, the top surface 70 of the needle guide body is slightly curved to assist with the placement of the needle 14 into the guide channel 64. The lengthwise dimension of the guide channel 64 allows the needle 14 to be inserted at various angles relative to the y axis of the needle guide assembly 10 as discussed more fully below. In a preferred form of the present embodiment, the bottom surface 72 of the needle guide body 60 is generally aligned and positioned planar with the bottom surface 28 of the body section 20. The location of the guide channel 64 extending below the bottom of the shorter side 22 of the body section 20 allows the needle 14 to be inserted at a shallower angle with respect to the tissue of the patient.

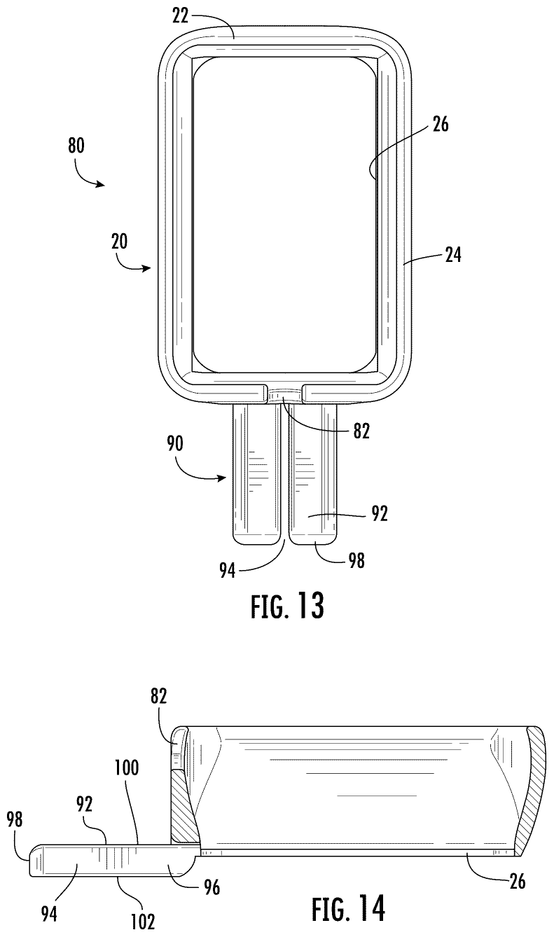

As shown in FIGS. 13-14, an alternate form of the needle guide assembly 80 of the present invention is disclosed. The needle guide assembly 80 of this embodiment generally includes a body section 20 and a needle guide body 90. In a preferred form of the present embodiment, the body section 20 is generally rectangular in shape to conform to the shape of the ultrasound probe 12. The body section 20 includes a pair of shorter length sides 22 and a pair of longer length sides 24. The shorter sides 22 correspond to the lateral side of the ultrasound probe 12 and the longer sides 24 of the body section 20 correspond to the transverse side of the ultrasound probe 12. The shorter side 22 of this embodiment that is adjacent to the needle guide body 90 includes an upper semicircular slot 82 to receive the probe indicator tab 27 from the ultrasound probe 12 therein. The body section 20 also includes a lip surface 26 that preferably extends along the entire lower surface of the ultrasound probe 12. The lip surface 26 engages the ultrasound probe 12 to allow the needle guide assembly 80 to be preferably aligned with the end of the ultrasound probe 12 for procedures where longitudinal access to the tissue of the patient is desired.

As shown, the needle guide body 90 of this embodiment is a generally U-shaped member that extends outwardly from the shorter side 22 of the body section 20. The needle guide body 90 includes a pair of elongate leg members 92 that extend generally from the midpoint of the shorter side 22 of the body section 20. The guide channel 94 extends between the leg members 92 and preferably has a width dimension that conforms to the desired needle size for use in the preferred procedure. The guide channel 94 starts at a first end 96 on the needle guide body 90 below the shorter side 22 of the body section 20. The guide channel 94 extends from the first end 96 to an open second end 98 that is spaced apart from the body section 20. The height or depth of the guide channel 94 between the top surface 100 of the needle guide body 90 and the bottom surface 102 of the needle guide body 90 is selected to limit side to side or lateral movement of a needle 14 along the z axis when a needle 14 is inserted into the guide channel 94. In this embodiment, the top surface 100 of the needle guide body is generally flat to limit lateral movement of the needle 14 and assist with the alignment of the needle 14 into the guide channel 94. The lengthwise dimension of the guide channel 94 allows the needle 14 to be inserted at various angles relative to the needle guide assembly 10 as discussed more fully herein. In a preferred form of the present embodiment, the bottom surface 102 of the needle guide body 90 is generally aligned and positioned below the bottom surface 28 of the body section 20 for situations where the end of the ultrasound probe 12 extends below the bottom surface 28 of the body section 20. The positioning of the guide channel 94 extending below the bottom of the shorter side 22 of the body section 20 also allows the needle 14 to be inserted at a shallower angle with respect to the tissue of the patient.

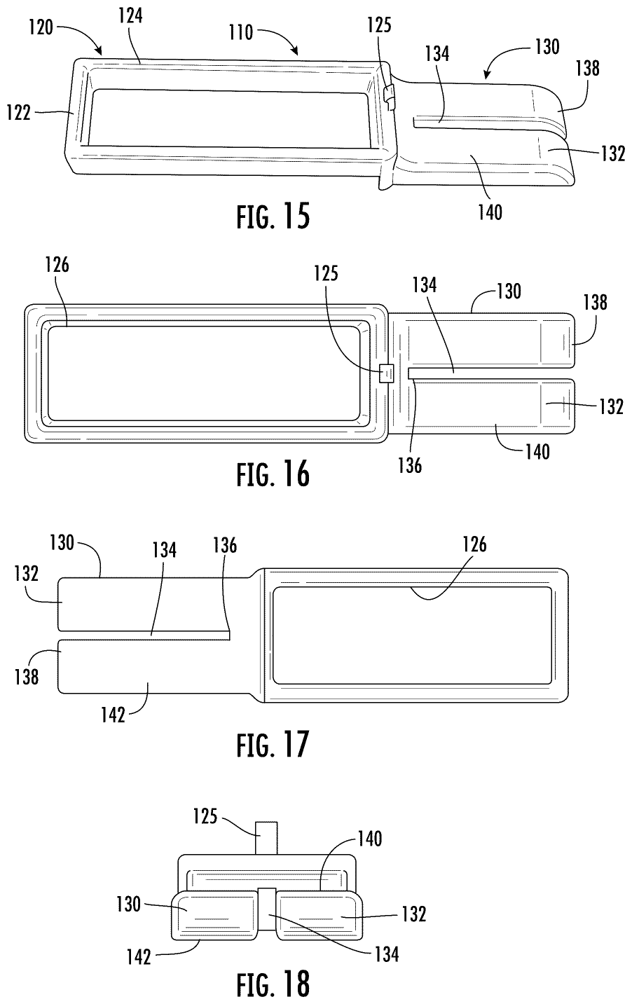

As shown in FIGS. 15-18, an alternate form of the needle guide assembly 110 of the present invention is disclosed. The needle guide assembly 110 of this embodiment generally includes a modified body section 120 and a needle guide body 130. In a preferred form of the present embodiment, the body section 120 is generally rectangular in shape to conform to the shape of an ultrasound probe 12. In this embodiment. The body section 120 has a more rectangular shape than the embodiments described above to allow this embodiment to attach to ultrasound probes having a different cross sectional shape. The body section 120 includes a pair of shorter length sides 122 and a pair of longer length sides 124. The shorter sides 122 correspond to the lateral side of the ultrasound probe 12 and the longer sides 124 of the body section 120 correspond to the transverse side of the ultrasound probe 12. The shorter side 122 of this embodiment adjacent to the needle guide body 130 includes an upper tab member 125 to engage with a portion of the ultrasound probe 12 such as the probe indicator 27. The body section 120 also includes a lip surface 126 that preferably extends along the entire lower surface of the ultrasound probe 12. The lip surface 126 engages the ultrasound probe 20 to allow the needle guide assembly 110 to be preferably aligned with the end of the ultrasound probe 12 for procedures where longitudinal access to the tissue of the patient is desired.

As shown, the needle guide body 130 of this embodiment is a generally elongated U-shaped member that extends outwardly from one of the shorter sides 122 of the body section 120. The needle guide body 130 includes a pair of elongate leg members 132 that extend generally from the midpoint of the shorter side 122 of the body section 120. The guide channel 134 extends between the leg members 132 and preferably has a width dimension that conforms to the desired needle size for use in the preferred procedure. The guide channel 134 starts at a first end 136 on the needle guide body 130 that is spaced apart from the shorter side 122 of the body section 120. The guide channel 134 extends from the first end 136 to an open second end 138 that is spaced apart from the body section 120. The height or depth of the guide channel 134 between the top surface 140 of the needle guide body 130 and the bottom surface 142 of the needle guide body 130 is selected to limit side to side or lateral movement of a needle 14 when a needle 14 is inserted into the guide channel 134. In this embodiment, the top surface 140 of the needle guide body is generally flat to limit lateral movement of the needle 14 and further includes tapered surfaces near the second end 138 assist with the alignment of the needle 14 into the guide channel 134. The increased lengthwise dimension of the guide channel 134 allows the use of a longer needle 14 to be inserted at various angles relative to the needle guide assembly 120 as discussed more fully below. In a preferred form of the present embodiment, the bottom surface 142 of the needle guide body 130 is generally aligned and positioned along the same plane as the bottom surface 128 of the body section 120.

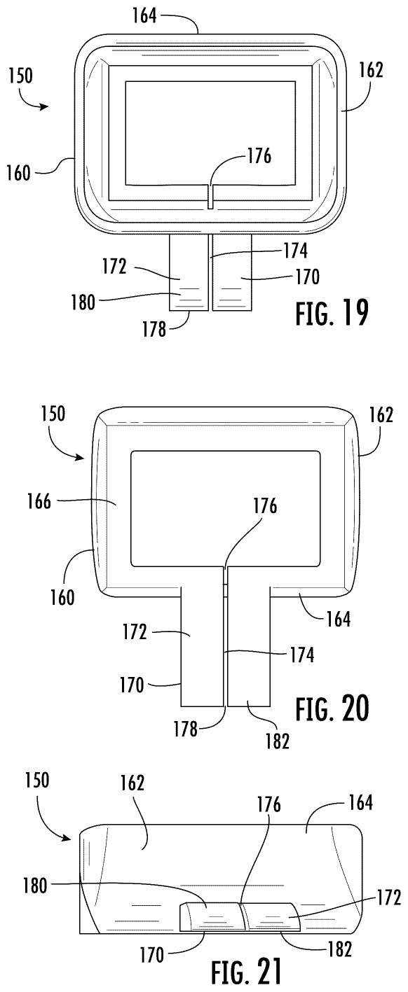

As shown in FIGS. 19-21, an alternate form of the needle guide assembly 150 of the present invention is disclosed. The needle guide assembly 150 of this embodiment generally includes a modified body section 160 and a needle guide body 170. In a preferred form of the present embodiment, the body section 160 is generally rectangular in shape to conform to the shape of an ultrasound probe 12. In this embodiment. The body section 160 has a generally rectangular shape as in the embodiments described above. The body section 160 includes a pair of shorter length sides 162 and a pair of longer length sides 164. The shorter sides 162 correspond to the lateral side of the ultrasound probe 12 and the longer sides 164 of the body section 160 correspond to the transverse side of the ultrasound probe 12. The body section 160 also includes a lip surface 166 that preferably extends along the entire lower surface of the ultrasound probe 12. In this embodiment, the lip surface 166 engages the ultrasound probe 12 to allow the needle guide assembly 150 to be preferably aligned with the end of the ultrasound probe 12 for procedures where transverse access to the tissue of the patient is desired.

As shown, the needle guide body 170 of this embodiment is a generally U-shaped member and extends outwardly from one of the longer sides 164 of the body section 160 to allow the user to visualize the target tissue from a transverse approach. The needle guide body 170 includes a pair of elongate leg members 172 that extend generally from the midpoint of one of the longer sides 164 of the body section 160 to allow the user to use a transverse needle insertion procedure. The guide channel 174 extends between the leg members 172 and preferably has a width dimension that conforms to the desired needle size for use in the preferred transverse procedure. The guide channel 174 starts at a first end 176 on the needle guide body 170 below the longer side 164 of the body section 160. The guide channel 174 extends from the first end 176 to an open second end 178 that is spaced apart from the body section 160. The height or depth of the guide channel 174 between the top surface 180 of the needle guide body 170 and the bottom surface 182 of the needle guide body 170 is selected to limit side to side or lateral movement of a needle 14 when a needle 14 is inserted into the guide channel 180. In this embodiment, the top surface 180 of the needle guide body is generally flat to limit lateral movement of the needle 14 and assist with the alignment of the needle 14 into the guide channel 174. The lengthwise dimension of the guide channel 174 allows the needle 14 to be inserted at various angles relative to the needle guide assembly 150 as discussed more fully below. In this embodiment, the thickness of the leg members 172 is greater near the first end 176 of the guide channel 174 than the thickness of the leg members 172 adjacent to the second end 178 of the leg members 172 to assist in the insertion of the needle 14 into the guide channel 174. In a preferred form of the present embodiment, the bottom surface 182 of the needle guide body 170 is generally aligned with the bottom surface 166 of the body section 160 for situations where the end of the ultrasound probe 12 extends generally flush with the bottom surface 166 of the body section 160. The positioning of the first end 176 of the guide channel 174 extending below the bottom of the longer side 164 of the body section 160 also allows the needle 14 to be inserted at a shallower angle with respect to the tissue of the patient and the placement of the needle guide body 170 adjacent to the longer side 164 of the body section 160 allows for the use of a transverse needle insertion approach. In the transverse approach, the view presented by the ultrasound probe is wider laterally and narrower depth wise than the longitudinal approach. Certain users prefer this approach to visualize certain types of tissue or procedures.

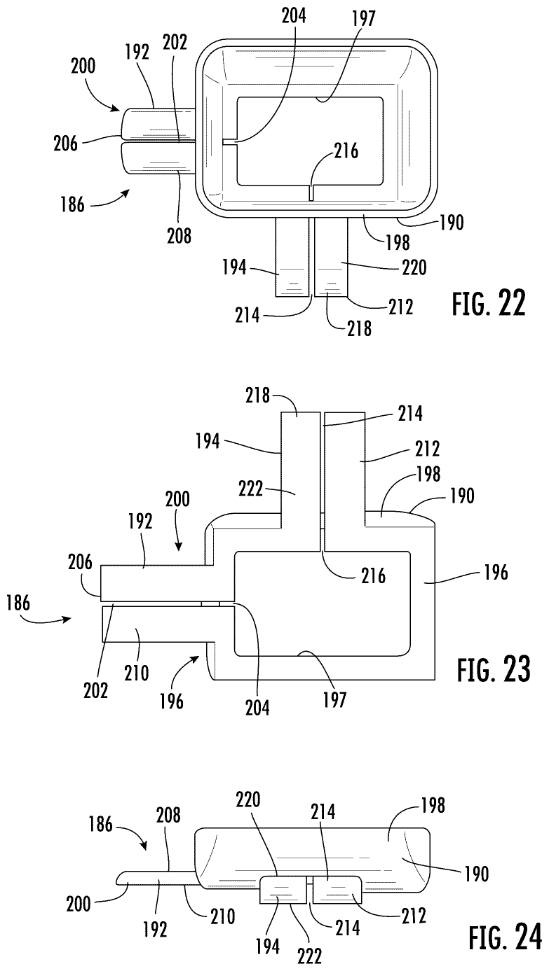

As shown in FIGS. 22-24, an alternate form of the needle guide assembly 186 of the present invention is disclosed. The needle guide assembly 186 of this embodiment includes a modified body section 190 and a needle guide body 200. In a preferred form of the present embodiment, the body section 190 is generally rectangular in shape to conform to the shape of an ultrasound probe 12. In this embodiment, the body section has two pairs of leg members 192 and 194. One of the shorter length sides 196 corresponds to the lateral side of the ultrasound probe 12 and includes a first pair of leg members 192 thereon. Additionally, one of the longer length sides 198 of the body section 190 engages and corresponds to the transverse side of the ultrasound probe 12 and includes a second pair of leg members 194 thereon. The body section 190 also includes a lip surface 197 that preferably extends along the entire lower surface of the ultrasound probe 12. The lip surface 197 engages the ultrasound probe 12 to allow the needle guide assembly 186 to be preferably aligned with the end of the ultrasound probe 12 for procedures where longitudinal and transverse access to the tissue of the patient is desired.

As shown, the needle guide assembly 186 of this embodiment includes a pair of generally elongated U-shaped members that extends outwardly from one of the shorter sides 196 and one of the longer sides 198 of the body section 190. The first leg members 192 of the first needle guide body 200 extend generally from the midpoint of the shorter side 196 of the body section 190. A first guide channel 202 extends between the first leg members 192 and preferably has a width dimension that conforms to the desired needle size for use in the preferred longitudinal procedure. The first guide channel 202 of the first needle guide body 200 starts at a first end 202 on the first needle guide body 200 and extends below the shorter side 196 of the body section 190. The first guide channel 202 extends from the first end 204 to an open second end 206 that is spaced apart from the body section 190. The height or depth of the first guide channel 202 between the top surface 208 of the first needle guide body 200 and the bottom surface 210 of the first needle guide body 200 is selected to limit side to side or lateral movement of a needle 14 when a needle 14 is inserted into the first guide channel 202. In this embodiment, the top surface 208 of the first needle guide body 200 is generally flat to further limit lateral movement of the needle 14 and additionally includes tapered surfaces near the second end 206 to assist with the alignment of the needle 14 into the first guide channel 202. The lengthwise dimension of the first guide channel 202 allows the use of a longer needle 14 to be inserted at various angles relative to the needle guide assembly. In a preferred form of the present embodiment, the bottom surface 210 of the first needle guide body 200 is generally aligned and positioned along the same plane as the bottom surface of the body section 190.

As shown, the needle guide assembly 186 of this embodiment also includes a pair of generally elongated U-shaped members that extends outwardly from one of the longer sides 198 of the body section 190. The second leg members 194 of the second needle guide body 212 extend generally from the midpoint of one of the longer sides 198 of the body section 190. A second guide channel 212 extends between the second leg members 194 and preferably has a width dimension that conforms to the desired needle size for use in the transverse procedure. The second guide channel 212 of the second needle guide body 214 starts at a first end 216 on the second needle guide body 214 and extends below the longer side 198 of the body section 190. The second guide channel 212 extends from the first end 216 to an open second end 218 that is spaced apart from the body section 190. The height or depth of the second guide channel 212 between the top surface 220 of the second needle guide body 214 and the bottom surface 222 of the second needle guide body 214 is selected to limit side to side or lateral movement of a needle 14 when a needle 14 is inserted into the second guide channel 212. In this embodiment, the top surface 220 of the second needle guide body 214 is generally flat to further limit lateral movement of the needle 14 and additionally includes preferably tapered surfaces near the second end 218 to assist with the alignment of the needle 14 into the second guide channel 212. The lengthwise dimension of the second guide channel 212 allows the use of a longer needle 14 to be inserted at various angles relative to the needle guide assembly. Additionally, in this embodiment, the width of the first guide channel 202 and the second guide channel 212 may be different from each other due to the differences in the typical angle of approach or needle sizes used in longitudinal and transverse approach procedures. Additionally, the length and thickness of the first leg members 192 and second leg members may be different due to the differences between a longitudinal and transverse procedure. This embodiment provides a versatile needle guide assembly 186 that may be used for multiple procedures and the preferences of the users so that a single needle guide assembly 186 may be adaptable to the needs of the user.

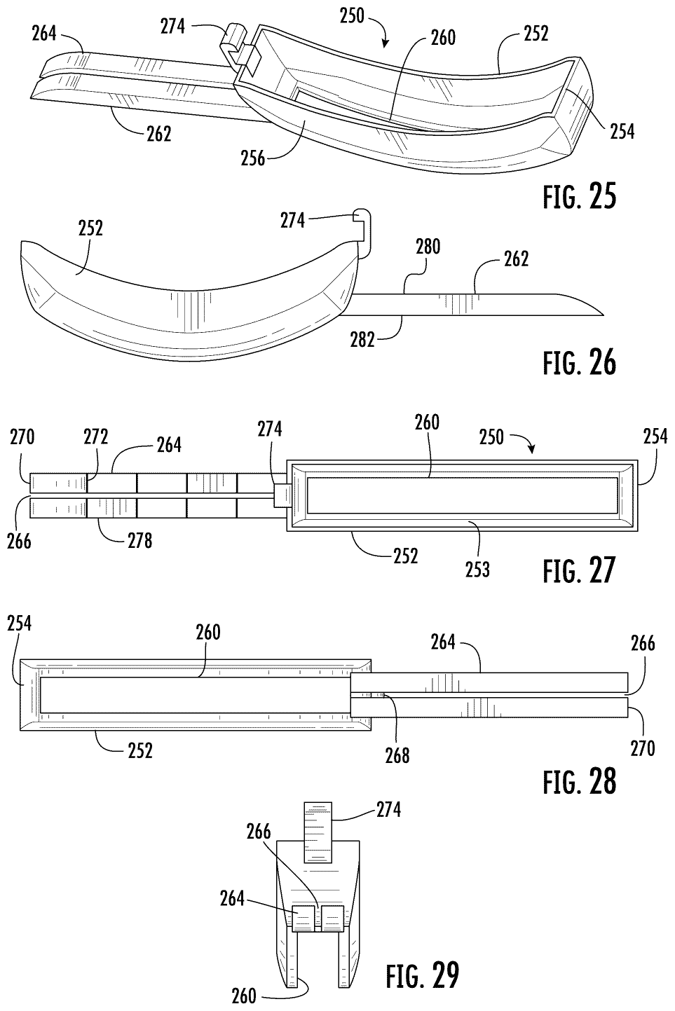

As shown in FIGS. 2A, 2B and 25-29, an alternate form of the needle guide assembly 250 of the present invention is disclosed. The needle guide assembly 250 of this embodiment generally includes a modified body section 252 and a needle guide body 262. In a preferred form of the present embodiment, the body section 252 is generally rectangular in shape to conform to the shape of a curvilinear ultrasound probe 12. In this embodiment. The body section 252 has a generally narrower and curved rectangular shape as compared to the embodiments described above. The body section 252 includes a pair of shorter length sides 254 and a pair of longer length sides 256. The shorter sides 254 correspond to the lateral side of the ultrasound probe 12 and the longer sides 256 of the body section 252 correspond to the transverse side of the ultrasound probe 12. The body section 252 also includes a lip surface 260 that preferably extends along the lower surface of the curvilinear ultrasound probe 12. In this embodiment, the lip surface 260 functions in combination with the indicator bracket 274 to secure and engage the ultrasound probe 12 to allow the needle guide assembly 250 to be preferably aligned with the end of the ultrasound probe 12 for procedures where longitudinal access to the tissue of the patient is desired. In this embodiment, the needle guide assembly 250 may be positioned so that the body section 252 and lip surface 260 are positioned adjacent to the face of the ultrasound probe 12 and the indicator bracket 274 engages the probe indicator 27 to provide a secure connection between the ultrasound probe 12 and the needle guide assembly 250.

As shown, the needle guide body 262 of this embodiment is a generally U-shaped member and extends outwardly from one of the shorter sides 254 of the body section 252 to allow the user to visualize the target tissue from a longitudinal approach. In FIG. 2B, a transverse guide body 278 is also provided. The transverse guide of FIG. 2B generally includes the features of the embodiment shown in FIGS. 19-21 and reference for these features are described above. The needle guide body 262 of this embodiment includes a pair of elongate leg members 264 that extend generally from the midpoint of one of the shorter sides 254 of the body section 252 to allow the user to use a longitudinal needle insertion procedure. The guide channel 266 extends between the leg members 264 and preferably has a width dimension that conforms to the desired needle size for use in the preferred longitudinal procedure. The guide channel 266 starts at a first end 268 on the needle guide body 262 below the shorter side 254 of the body section 252. The guide channel 266 extends from the first end 268 to an open second end 270 that is spaced apart from the body section 252. The height or depth of the guide channel 266 between the top surface 280 of the needle guide body 262 and the bottom surface 282 of the needle guide body 262 is selected to limit side to side or lateral movement of a needle 14 when a needle 14 is inserted into the guide channel 266. In this embodiment, the top surface 280 of the needle guide body is generally flat to limit lateral movement of the needle 14 and assist with the alignment of the needle 14 into the guide channel 266. The lengthwise dimension of the guide channel 266 allows the needle 14 to be inserted at various angles relative to the needle guide assembly 250 as shown in FIGS. 2A and 2B. In this embodiment, the thickness of the leg members 264 is greater near the first end 268 of the guide channel 266 than the thickness of the leg members 264 adjacent to the second end 270 of the leg members 264 to assist in the insertion of the needle 14 into the guide channel 266. In a preferred form of the present embodiment, the bottom surface 282 of the needle guide body 262 is generally adjacent to the bottom surface 253 of the body section 252 to accommodate the shape of the curvilinear ultrasound probe 12. The positioning of the first end 268 of the guide channel 266 extending adjacent to the bottom of the shorter side 254 of the body section 252 also allows the needle 14 to be inserted at a shallow angle with respect to the tissue of the patient and the placement of the needle guide body 262 adjacent to the shorter side 254 of the body section 252 allows for the use of a longitudinal needle insertion approach. In the longitudinal approach, the view presented by the ultrasound probe is wider depth wise and narrower laterally than the transverse approach. Certain users prefer this approach to visualize certain types of tissue or procedures.

Another aspect of this design shown in FIGS. 2A, 2B and FIGS. 25-29 is the optional inclusion of the needle guide rulers 272 and angle guide 276 along the guide channel 266 to observe how far the needle entry is from the center of the probe transducer as well as lines inside of the angle guide 276 showing a 45 degree angle of approach. Additionally, as shown in FIG. 2C, the use of the open ended guide channel allows the user to lift the needle guide assembly 250 and ultrasound probe 12 from the skin surface of the patient and insert a catheter over the needle as seen in USGN catheter cannulation oblique front view” and “USGN catheter insertion oblique front view”. This type of procedure is not available in devices that use enclosed sleeves for the needle. Furthermore, as shown in FIG. 2B, the needle guide assembly 250 may include guide channels for longitudinal and transverse use. This versatility allows a variety of insertion angles into the patient's body and allows the user to insert the needle using the transverse view of the ultrasound probe for an out of plane technique or the longitudinal view of the ultrasound probe for an in plane technique while the guide channel ensures that the needle is centrally positioned in the visible plane of the ultrasound probe.

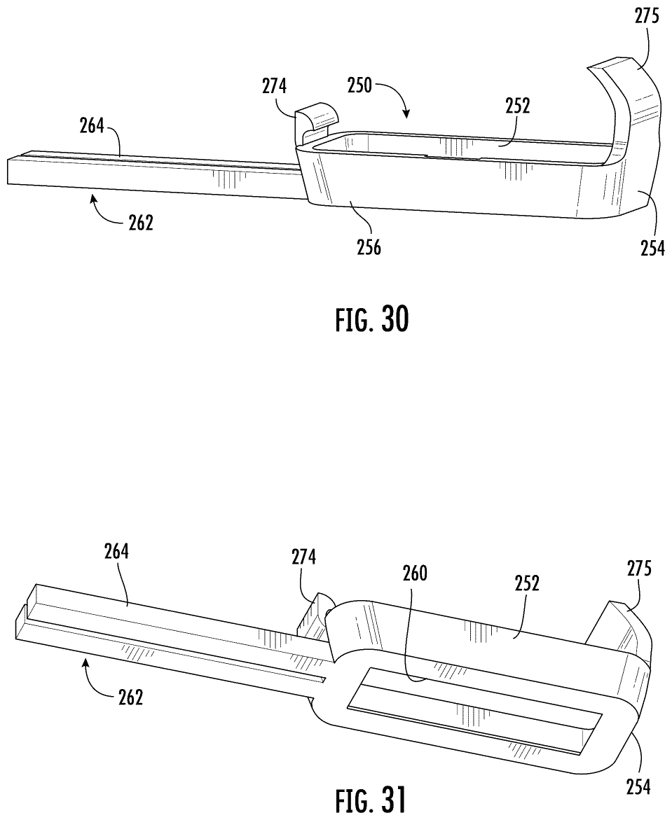

FIGS. 30 and 31 are illustrative of an alternate embodiment of the present invention. In this embodiment, like numbers are added to like elements described herein. The needle guide assembly 250 of this embodiment generally includes a modified body section 252 and a needle guide body 262. In a preferred form of the present embodiment, the body section 252 is generally rectangular in shape to conform to the shape of a curvilinear ultrasound probe 12. In this embodiment. The body section 252 has a generally narrower and curved rectangular shape as compared to the embodiments described above. The body section 252 includes a pair of shorter length sides 254 and a pair of longer length sides 256. The shorter sides 254 correspond to the lateral side of the ultrasound probe 12 and the longer sides 256 of the body section 252 correspond to the transverse side of the ultrasound probe 12. The body section 252 also includes a lip surface 260 that preferably extends along the lower surface of the curvilinear ultrasound probe 12. In this embodiment, the lip surface 260 functions in combination with the indicator bracket 274 and a back retention bracket 275 to secure and engage the ultrasound probe 12 to the body section 252. This secure attachment allows the needle guide assembly 250 to be preferably aligned with the end of the ultrasound probe 12 for procedures where longitudinal access to the tissue of the patient is desired. In this embodiment, the needle guide assembly 250 may be positioned so that the body section 252 and lip surface 260 are positioned adjacent to the face of the ultrasound probe 12 and the combination of the indicator bracket 274 and retention bracket 275 engage the probe indicator 27 and the body of the probe 12 to provide a secure connection between the ultrasound probe 12 and the needle guide assembly 250.

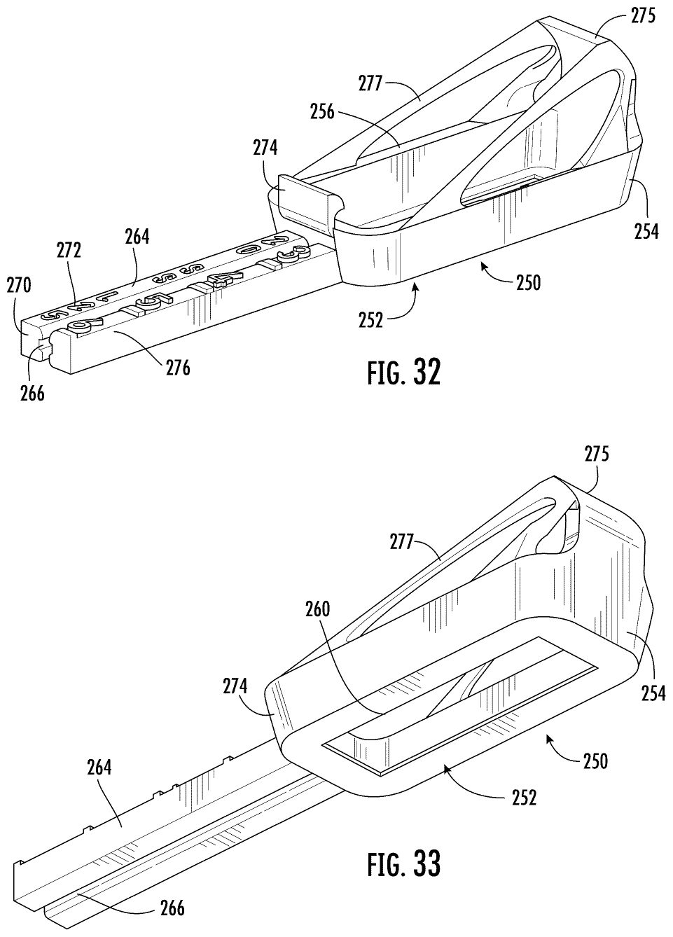

FIGS. 32 and 33 are illustrative of another alternate embodiment of the present invention. In this embodiment, like numbers are added to like elements described herein. The needle guide assembly 250 of this embodiment generally includes a modified body section 252 and a needle guide body 262. In a preferred form of the present embodiment, the body section 252 is generally rectangular in shape to conform to the shape of a curvilinear ultrasound probe 12. In this embodiment. The body section 252 has a generally narrower and curved rectangular shape as compared to the embodiments described above. The body section 252 includes a pair of shorter length sides 254 and a pair of longer length sides 256. The shorter sides 254 correspond to the lateral side of the ultrasound probe 12 and the longer sides 256 of the body section 252 correspond to the transverse side of the ultrasound probe 12. The body section 252 also includes a lip surface 260 that preferably extends along the lower surface of the curvilinear ultrasound probe 12. In this embodiment, the lip surface 260 functions in combination with the indicator bracket 274 and a back retention bracket 275 to secure and engage the ultrasound probe 12 to the body section 252. This embodiment further includes a pair of flexible band elements 277 extending from a location generally adjacent to the retention bracket 275. The band elements 277 enable the body section 252 to be attachable to a variety of sizes and shapes of ultrasound probes 12 by increasing retention pressure between the body section 252 and the ultrasound probe 12. This secure attachment allows the needle guide assembly 250 to be preferably aligned with the end of the ultrasound probe 12 for procedures where longitudinal access to the tissue of the patient is desired. In this embodiment, the needle guide assembly 250 may be positioned so that the body section 252 and lip surface 260 are positioned adjacent to the face of the ultrasound probe 12 and the combination of the indicator bracket 274, retention bracket 275 and band elements 277 engage the probe indicator 27 and the body of the probe 12 to provide a secure connection between the ultrasound probe 12 and the needle guide assembly 250.

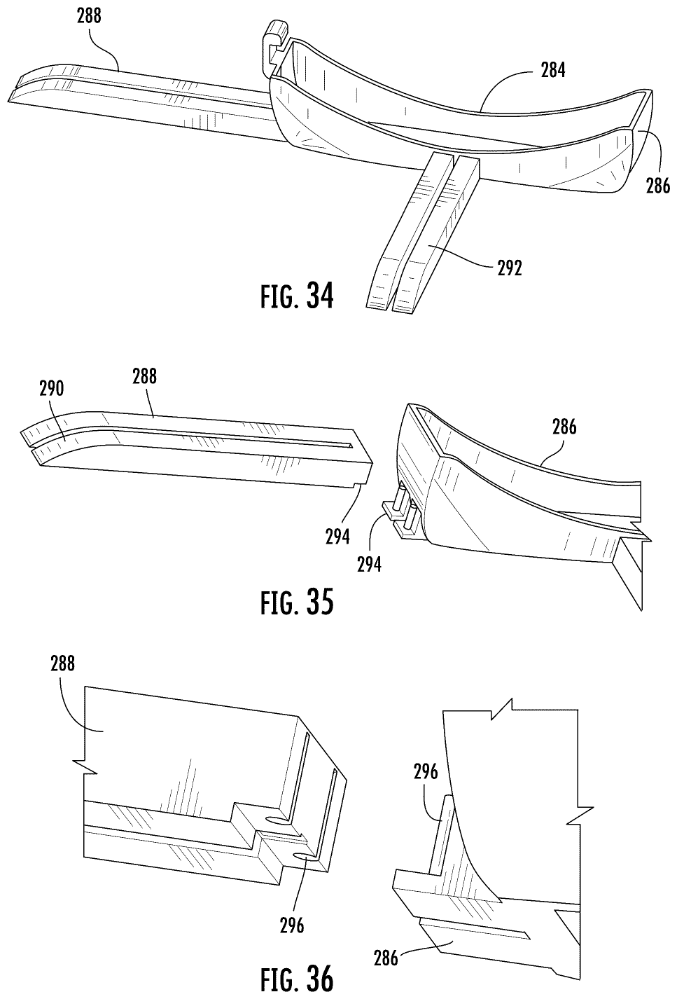

FIGS. 34-36 are illustrative of another embodiment of the present invention wherein a modified needle guide assembly 284 includes a modified body section 286 and a modified needle guide body 288. As shown in FIG. 34, the needle guide body 288 is releasably attachable to the body section 286. This enables the user to attach the needle guide body 288 to the body section in either the lateral or transverse positions. FIG. 35 illustrates the first method of attachment between the body section 286 and the needle guide body 288 wherein multiple complementary snap connectors 294 are used to securely and reliably connect the body section 286 and needle guide body 288. As shown in FIG. 36, peg and post connectors 296 may also be used to connect the body section 286 to the needle guide body 288. The use of the peg and post connectors 296 allows the user to slide the needle guide body 288 downwardly along the post section of the connectors on the body section 286 to provide a secure connection therebetween. The connection mechanism in which these components are fastened may change and can include but not limited to a latch, clip, push fit or screws. The body section 286 and needle guide body 288 shown in FIGS. 34-36 include the features and functions described above with respect to the embodiments described above. This attachment system allows the user to interchange needle guide body 288 having a different width or style to accommodate various sized needles without having to change the body section.

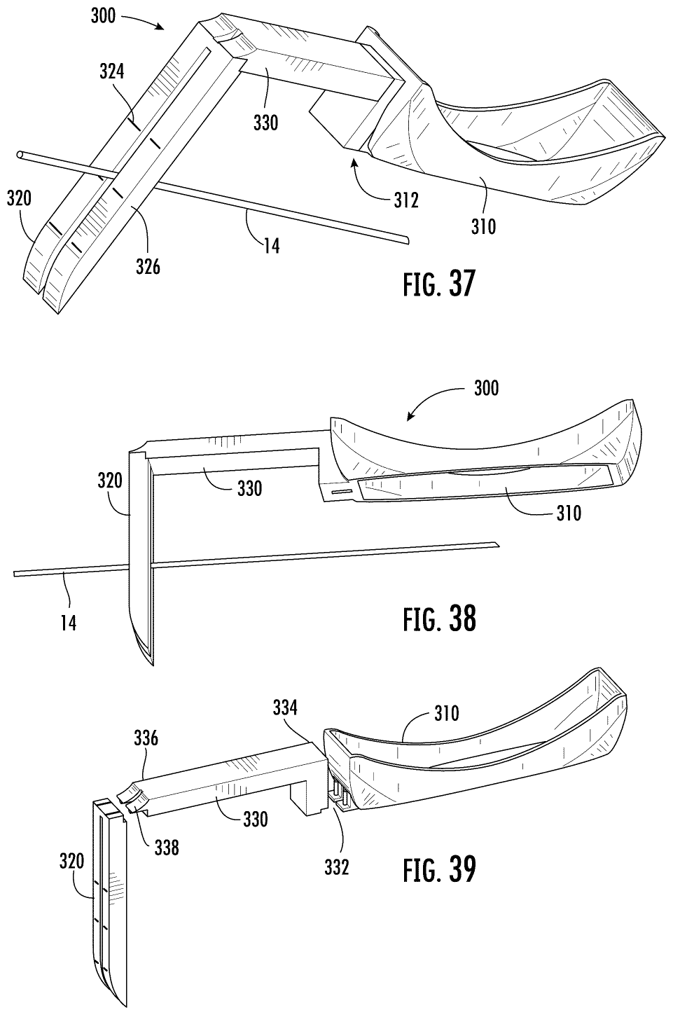

FIGS. 37-39 are illustrative of a further embodiment of the present invention. The needle guide assembly 300 allows the operator to allow needle entry at or near 0 degrees to the face of the ultrasound probe improving needle visualization and may be better suited for specific procedures where the needle entry is at a distance or on the side of procedural area (IE: Forearm nerve blocks, RAPTIR (Retroclavicular approach to an infraclavicular brachial plexus block) nerve block. The needle guide assembly 300 of this embodiment includes a body section 310 and a needle guide body 320. Each of these elements are similar and/or interchangeable with the embodiments described above and therefore are not separately repeated here. An extender element 330 is provided which interconnects between the body section 310 and the needle guide body 320. The extender 330 is an elongate and generally flat element that includes a first connector 332, such as a snap connector, peg and post connector or similar connectors on the first end 334 thereof. The first connector releasably engages a complementary connector 312 on the body section. The second end 336 of the extender 330 includes a second connector 338 thereon. The second connector 338 releasably engages a complementary connector 322 on the needle guide body 320.

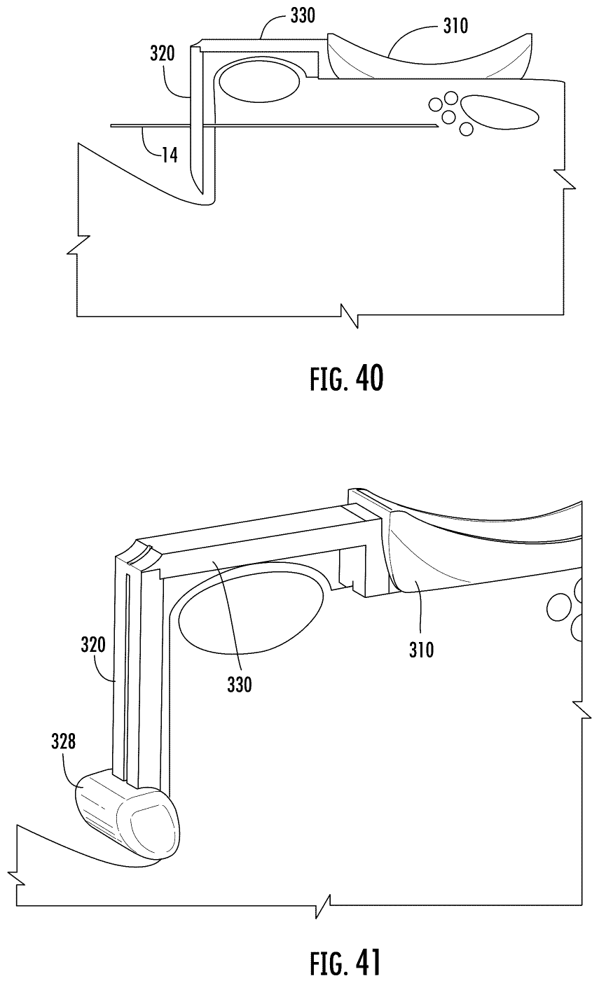

In this embodiment, the extender 330 may be of a variety of lengths, sizes, designs and/or curvatures to allow the user to perform the procedure along various anatomical structures. The extender 330 can allow the user to avoid certain structures that are higher than the ultrasound probe transducer face (IE: Clavicle). FIGS. 40 and 41 illustrate the use of the needle guide assembly 300 in a specific procedure where a near 0 degree needle entry is necessary and where the clavicle may necessitate the need for a vertically offset extender. In this embodiment, the extender 330 allows the needle guide body 320 to be spaced apart from the body section 310 such that the clavicle of the patient is positioned adjacent to the body section 310 and above the needle guide body 320 to allow the user to utilize a zero angle approach with the needle 12 to the target tissue. As shown in FIG. 37, the needle guide body 320 may include a needle guide ruler 324 to provide the user with a visual indication of the distance. In this procedure, the needle guide ruler 324 provides the user with a visual confirmation that the intended needle insertion location. The needle guide body 320 of this embodiment also preferably includes an angle guide 326 to provide the user with an indication of the angle of entry for the needle 14. In this embodiment, the angle guide 326 is preferably a zero degree angle of entry.

As shown in FIG. 41, the needle guide body 320 may include a second comfort end 328 that is configured to provide a cushioned surface against the tissue of the patient. The use of the comfort end 328 also provides a further source of support to ensure that the needle guide assembly 300 is stable and non-moving during the procedure.

The foregoing embodiments are illustrative examples of the present invention. It is intended that variations may be made to the specific structures described above and illustrated in the attached drawings. The scope of the present invention is intended to be defined by the attached claims.

1. A guidance device for a needle guide assembly, said
device including a body section and a needle guide assembly,
said body section including a top and bottom surface and a longitudinal dimension and a transverse dimension formed by a plurality of sidewalls configured to receive a forward looking imaging device therein;
said needle guide assembly including a pair of elongate leg members having first and second ends and top and bottom surfaces and extending from a sidewall of the body section and wherein the bottom surface of the leg members are generally aligned with the bottom surface of the body section: and
a guide channel extending between said leg members wherein said guide channel incudes a first and second end with the first end positioned adjacent to said sidewall of the body section and said second end of said guide channel extends to said second end of said leg member and said guide channel forming an opening extending lengthwise along the leg members and between said top and bottom surfaces of said leg members and wherein said guide channel is configured to receive a needle extending therethrough between the top and bottom surfaces thereof.

2. The guidance device of claim 1 wherein at least one the leg member includes a distance indicator thereon to indicate the distance from the body section along the leg member.

3. The guidance device of claim 1 wherein at least one of the leg members include an angle indicator to indicate a desired angle relative to the lengthwise dimension of the leg members.

4. The guidance device of claim 1 wherein the leg members extend from the longitudinal dimension of the body section.

5. The guidance device of claim 1 wherein the leg members extend from the transverse dimension of the body section.

6. The guidance device of claim 1 wherein a first pair of leg members extend from the longitudinal dimension of the body section and a second pair of leg members extend from the transverse dimension of the body section and wherein said first and second pairs of leg members each include a guide channel therein.

7. The guidance device of claim 1 wherein the sidewalls of the body section includes a pair of short sidewalls and a pair of elongate sidewalls wherein the short sidewalls are generally parallel to each other and the long sidewalls are generally parallel to each other and form an open area therebetween that is configured to allow the insertion of the forward looking imaging device therein and a lip member extending inwardly along a plurality of the sidewalls to contact and engage the forward looking imaging device.

8. The guidance device of claim 7 wherein the body section includes a tab member extending inwardly from at least one of the sidewalls and said tab member is configured to engage at least a portion of the forward looking imaging device.

9. The guidance device of claim 8 wherein the body section further includes a retention bracket that is configured to engage at least a portion of the forward looking imaging device.

10. The guidance device of claim 7 wherein the body section further includes at least one flexible band element configured to releasably engage the forward looking imaging device.

11. The guidance device of claim 1 wherein the leg members are releasably attached to the body section of the guidance device.

12. The guidance device of claim 1 further including an elongate extender detachably positioned between the body section and the leg members wherein the extender includes top and bottom surfaces and wherein said bottom surface is generally aligned with the bottom surface of the body section.

13. The guidance device of claim 12 wherein the bottom surface of the leg members are generally oriented with the bottom surface of the body section in a non parallel orientation.

14. A guidance device for a needle guide assembly, said device including a body section and a needle guide assembly;
said body section including a top and bottom surface and a longitudinal dimension and a transverse dimension formed by a plurality of sidewalls configured to receive a forward looking imaging device therein;
wherein the sidewalls of the body section includes a pair of shorter length short sidewalls and a pair of elongate longer length sidewalls wherein the short sidewalls are generally parallel to each other and the long sidewalls are generally parallel to each other and form an open area therebetween that is configured to allow the insertion of the forward looking imaging device therein and a lip member extending inwardly along a plurality of the sidewalls to contact and engage the forward looking imaging device;
said needle guide assembly including a pair of elongate leg members having first and second ends and top and bottom surfaces and extending from a sidewall of the body section and wherein the bottom surface of the leg members are generally aligned with the bottom surface of the body section; and
a guide channel extending between said leg members wherein said guide channel incudes a first and second end with the first end positioned beneath said sidewall of the body section and said second end of said guide channel extends to said second end of said leg member and said guide channel forming an opening extending lengthwise along the leg members and between said top and bottom surfaces of said leg members and along the entire distance between the first and second ends thereof.

15. The guidance device of claim 14 wherein at least one the leg members includes a distance indicator thereon to indicate the distance from the body section along the leg member and at least one of the leg members include an angle indicator to indicate a desired angle relative to the lengthwise dimension of the leg members.

16. The guidance device of claim 14 further including an elongate extender detachably positioned between the body section and the leg members wherein the extender includes top and bottom surfaces and wherein said bottom surface is generally aligned with the bottom surface of the body section and wherein the bottom surface of the leg members are generally oriented with the bottom surface of the body section in a non parallel orientation.

17. The guidance device of claim 14 wherein the body section includes a tab member extending inwardly from one of the short sidewalls and said tab member is configured to engage at least a portion of the forward looking imaging device and further including a retention bracket that is configured to engage at least a portion of the forward looking imaging device.

18. The guidance device of claim 14 wherein the leg members extend from one of the short sidewalls of the body section along the longitudinal dimension of the body section.

19. A guidance device for a needle guide assembly,
said device including a body section and a needle guide assembly;
said body section including a top and bottom surface and a longitudinal dimension and a transverse dimension formed by a plurality of sidewalls configured to receive a forward looking imaging device therein;
wherein the sidewalls of the body section includes a pair of shorter length short sidewalls and a pair of elongate longer length sidewalls wherein the short sidewalls are generally parallel to each other and the long sidewalls are generally parallel to each other and form an open area therebetween that is configured to allow the insertion of the forward looking imaging device therein and a lip member extending inwardly along a plurality of the sidewalls to contact and engage the forward looking imaging device;
said needle guide assembly including a pair of elongate leg members having first and second ends and top and bottom surfaces and extending from a sidewall of the body section and wherein the bottom surface of the leg members are generally aligned with the bottom surface of the body section and the leg members are detachable from the body section: and
a guide channel extending between said leg members wherein said guide channel incudes a first and second end with the first end positioned beneath said sidewall of the body section and said second end of said guide channel extends to said second end of said leg member and said guide channel forming an opening extending lengthwise along the leg members and between said top and bottom surfaces of said leg members and along the entire distance between the first and second ends thereof.

20. The guidance device of claim 19 wherein a first pair of leg members extend from the longitudinal dimension of the body section and a second pair of leg members extend from the transverse dimension of the body section and wherein said first and second pairs of leg members each include a guide channel therein.
